SEQUENCE LISTING (2 sequences)

SEQ ID NO: 1 - DNA, 48 bp, Artificial Sequence
  Description of Artificial Sequence DNA Allosteric probe
cctggggagtattgcggaggaagggcctgggggtattgcggaggaagg

SEQ ID NO: 2 - RNA, 85 nt, Artificial Sequence
  Description of Artificial Sequence RNA Allosteric probe
gcuuaauacgacucacuauaggccugggcgagaaguuuaggccuuggguugggaagaaacuguggcacuucggugccagcaaccc

(19) United States
(12) Patent Application Publication
(10) Pub. No.: US 2005/0026178 A1
(43) Pub. Date: Feb. 3, 2005

(54) Allosteric probes and methods

(76) Inventor: Marit Nilsen-Hamilton, Ames, IA (US)

Correspondence Address: DICKSTEIN SHAPIRO MORIN & OSHINSKY LLP, 2101 L Street, NW, Washington, DC, 20037, US

(21) Appl. No.: 10/809,886

(22) Filed: Mar. 26, 2004

Related U.S. Application Data
(60) Provisional application No. 60/457,936, filed on Mar. 28, 2003.

(51) Int. Cl. C12Q 1/68; C07H 21/04

(52) U.S. Cl. 435/6; 536/24.3

(57) ABSTRACT

The present invention provides allosteric probes and methods of using allosteric probes for binding a first target and at least a second target. The probes have an allosteric molecule and at least one regulated aptamer. The binding of the allosteric molecule to a first target enhances or inhibits the binding of the at least one regulated aptamer to at least a second target. The binding of the at least one regulated aptamer to its target can be used to detect the presence of a particular target or targets or to selectively target a prodrug, drug, or toxin to a target cell or group of cells. The allosteric probes have a wide variety of diagnostic and therapeutic applications.

### CROSS REFERENCE TO OTHER APPLICATIONS

This patent application claims the benefit of U.S. provisional patent application Ser. No. 60/457,936, filed Mar. 28, 2003, and U.S. provisional patent application Ser. No. 60/198,370, filed May 2, 2003, the entirety of which are hereby incorporated by reference.

### REFERENCES

Several publications are referenced herein. Full citations for these publications are provided below. The disclosures of these publications are incorporated herein by reference in their entirety, unless otherwise noted.

### BACKGROUND OF THE INVENTION

Detection and treatment of disease is a cornerstone of biotechnology and molecular biology. Accurate and rapid detection of disease is increasingly important as the prevalence of pathogenic organisms and threat of bioterrorism increases. Treatment of diseases, such as cancer, requires the ability to specifically deliver drugs and toxins to diseased cells while leaving normal cells unharmed. Nucleic acids, antibodies and other biomolecules should be engineered to provide improved specificity and activity in both the detection and treatment of disease.

Nucleic acid probes are typically short nucleic acid sequences used to detect, amplify, and quantify DNA and RNA for diagnostic and therapeutic applications. In particular, nucleic acid probes are designed to specifically hybridize with complementary target nucleic acid sequences. For diagnostic applications, nucleic acid probes can be labeled (e.g., with radioactive or fluorescent tags) in order to detect the presence or absence of the target nucleic acid sequence using a variety of techniques (e.g., fluorescent in-situ hybridization, Southern blot, Northern blot, and chromatography). While these techniques are useful for detecting the presence or absence of particular target sequences, their sensitivity depends on the amount of target nucleic acid present in the sample. Furthermore, living cells cannot be analyzed using these techniques since samples must be extracted, and fixed or frozen prior to analysis.

In order to increase the sensitivity of nucleic acid detection techniques, labeled probes can be combined with amplification processes, such as PCR, to amplify and detect nucleic acids. The combination of probes and amplification processes has been especially useful in forensic applications where the nucleic acid of interest is present in extremely small quantities. Many disease conditions are diagnosed by comparing the amount of mRNA produced in a normal cell to a diseased cell. For example, increased expression of an oncogene may indicate the presence of a tumor cell. Amplification techniques, such as PCR, are non-linear and exponentially increase the amount of nucleic acid present in a sample. Thus, the amount of nucleic acid present in a sample subject to PCR is not representative of the amount of nucleic acid originally present in the sample and such techniques are of limited use for quantification purposes.

Previously, nucleic acids were generally thought to be useful solely for storing, transporting, processing, and expressing genetic information. Recently, however, nucleic acids capable of additional functions have been identified. These “functional nucleic acids” can, for example, catalyze reactions or bind specifically to particular targets. The three-dimensional structure of functional nucleic acids provides the specificity necessary to bind other compounds, much like the three-dimensional structure of an enzyme determines its specificity for a substrate. The small size, specificity, and ease of manipulation of nucleic acids can be applied to functions traditionally associated with proteins (e.g., catalysis, receptors, and antibodies).

Molecular beacons are functional single-stranded DNA probes that can report the presence of specific nucleic acids. Molecular beacons are stem-loop shaped molecules containing a nucleotide sequence in the loop portion of the molecule complementary to a target DNA or RNA. Molecular beacons are labeled on one end with a fluorescent molecule and on the other with a quenching molecule. In its native hairpin structure, the quenching molecule is in close proximity with the fluorescent molecule and absorbs the light emission of the fluorescent molecule. When the complementary nucleotide sequence on the molecular beacon loop binds its target molecule, the molecular beacon undergoes a conformational change that opens up the stem-loop structure and moves the fluorescent molecule and the quenching molecule away from each other. The light emission from the fluorescent molecule is no longer quenched and the signal can be detected.

Molecular beacons are of limited use in generating amplified signals. The fluorescent signal of a molecular beacon is an integral part of the molecular beacon. Thus, the fluorescent molecule of the molecular beacon can generate only one signal in the presence of its target. The signal cannot be further amplified or altered by use of, for example, secondary labeled antibodies. In addition, the weak, unamplified signal generated by the molecular beacon is not able to penetrate living tissue sufficiently for use in non-invasive imaging.

Aptamers are functional synthetic nucleic acids useful for high-affinity binding to targets (e.g., nucleic acids, proteins, and chemical compounds). Unlike naturally occurring nucleic acids, which transfer genetic information, aptamers are selected on the basis of their ability to specifically bind their ligand. Systematic Evolution of Ligands by Exponential Enrichment (SELEX) is a process of selecting aptamers directed to a chosen ligand. See Hermann and Patel, Science 287, pp 820-825 (2000); U.S. Pat. Nos. 5,475,096, 5,595,877, 5,660,985, and 6,180,348, hereby incorporated by reference in their entirety. The SELEX process selects aptamers by screening random sequence libraries, retaining sequences that bind the chosen target molecule, and repeating the cycle with increasing levels of binding stringency. The selected aptamer is capable of binding a chosen target but not other molecules.

Ribozymes are functional nucleic acid molecules with enzymatic capabilities, including the ability to cleave nucleic acid molecules in a sequence-specific manner. The three-dimensional structure of a ribozyme, like a protein or enzyme, determines the specificity of its interaction with a particular target. Allosteric ribozymes are ribozyme nucleic acid constructs having a ribozyme portion and an antisense nucleic acid portion or a small molecule binding portion. The activation of the ribozyme is regulated by the binding of the antisense nucleic acid to a complementary nucleic acid target or by binding of the small molecule to the small molecule binding portion. In the absence of the antisense target, the ribozyme is inactive. In the presence of the antisense target, the conformation of the allosteric ribozyme is perturbed resulting in activation of the ribozyme so that it can catalyze a reaction.

Allosteric ribozymes regulate the rate of a reaction catalyzed by the ribozyme portion of the molecule. For example, a reaction catalyzed by a ribozyme can be represented as A+B [symbol] AB [symbol] A+C. The regulatory portion of the allosteric ribozyme (i.e., antisense molecule, small molecule binding molecule) regulates the second step of this reaction (e.g., AB [symbol] A+C) rather than the first step of the reaction (A+B [symbol] AB). Thus, allosteric ribozymes are not designed to overcome the energy barriers to the transition between certain conformational states. In other words, the allosteric ribozyme does not drive the formation of AB from A+B. Rather, allosteric ribozymes increase the rate of the catalysis of AB into A+C.

The specificity of aptamers, ribozymes, and other nucleic acid probes is also limited by non-specific binding. Nucleic acid probes and other binding molecules can non-specifically bind to many targets albeit at a lower affinity than the probe's specific target. This non-specific binding can interfere with and significantly lower the specificity of the nucleic acid probe binding to its intended target.

Treatment and detection of cancer is most effective if the active agent (e.g., drug, toxin, or detection molecule) is targeted directly to the cancer cells. Treatment can also be greatly facilitated by noninvasive in vivo imaging techniques. The most effective technologies should combine therapy and imaging in a seamless transition. Monoclonal antibodies have shown promise for specific cell targeting and have been applied to both therapy and imaging. Many humanized monoclonal antibodies are currently approved for clinical use or are currently in clinical trials. However, most monoclonal antibodies have proven to be minimally effective in treating cancers and are generally used clinically in combination with other treatments due to their large size and poor penetration ability. Directly arming mAbs with toxins results in more potent tumor killing than antibody alone. However, this approach suffers from high systemic toxicity. The high toxicity is likely the result of many nonspecific interactions between mAbs and other cellular macromolecules. Thus, there is a need for therapies with higher specificity for cancer cells and which do not damage normal cells.

### BRIEF SUMMARY OF THE INVENTION

In a preferred embodiment, the present invention provides allosteric probes comprising an allosteric regulator and at least one regulated aptamer. The binding of the allosteric regulator to a first target enhances the binding of the at least one regulated aptamer to at least one second target. The modular nature of the allosteric probes provides flexibility in designing custom probes using various combinations of allosteric regulators and regulated aptamers.

The allosteric regulator is preferably linked to the at least one regulated aptamer. When the allosteric regulator binds to a first target, the binding of the at least one regulated aptamer to the second target is enhanced. For example, when the allosteric regulator binds to its target, the conformation of the regulated aptamer is altered, enhancing the ability of the regulated molecule to bind to its target.

In another embodiment of the invention, a method of detecting a target with a probe comprising an allosteric regulator linked to at least one regulated aptamer is provided. In a preferred method, the probe is contacted with a first target. The contacting of the allosteric regulator with the first target enhances the binding of the at least one regulated aptamer to at least one second target, producing a detectable signal (e.g., fluorescent label, or radioactive label). Preferably, the affinity of the allosteric regulator for its target is greater than the affinity of the regulated aptamer for its target. Preferably, the allosteric regulator has a relative affinity (e.g., as measured by its Kd) for its target that is at least about 1 log higher than the affinity of the regulated aptamer for its target.

The invention also provides preferred methods of selectively targeting, accumulating or localizing, a drug, prodrug, or toxic agent to or in the proximity of a target cell or a group of cells (e.g., tumor cells, infected cells, or microorganisms) by providing an allosteric probe comprising an allosteric regulator linked to at least one regulated aptamer. In this embodiment, the binding of a cell to the allosteric regulator enhances the binding of the regulated aptamer to the drug, prodrug, or toxic agent, increasing the concentration of the toxic agent in the proximity of the target cell or group of cells and maintaining the toxic agent where it is needed for therapeutic purposes, thus enhancing therapeutic applications and reducing systemic side effects. Preferably, the drug, prodrug, or toxic agent is activated only when it is in proximity to the target cell (e.g., the drug, prodrug, or toxic agent is activated after being cleaved or modified by an enzyme secreted by the target cell). The selective targeting or accumulation of a drug, prodrug, or toxic agent in proximity to the desired cell or group of cells has a beneficial therapeutic effect and/or results in cell damage, inactivation, or death.

The above and other characteristics and advantages of the invention can be better understood from the following written description and the accompanying drawings or may be learned by practice of the invention.

### BRIEF DESCRIPTION OF THE DRAWINGS

FIG. 1 depicts an exemplary allosteric probe including an allosteric regulator and a regulated aptamer.

FIG. 2 shows the regulation of an exemplary allosteric probe comprising a neomycin aptamer (e.g., allosteric regulator) linked to an ATP aptamer (e.g., regulated aptamer). In this example, the activity of the ATP aptamer, as measured by its ability to bind ATP, is enhanced in the presence of neomycin.

FIGS. 3A-3C depict an exemplary allosteric probe comprising two aptamers linked in cis. One aptamer (PSCA aptamer) has a high affinity for prostate stem cell antigen (“PSCA”), a marker for metastatic prostate cancer cells. A second aptamer (inulin aptamer) has an affinity for inulin (a fructan carbohydrate) that is lower than the affinity of the PSCA aptamer for PSCA. In the presence of PSCA, the PSCA aptamer (e.g., the allosteric regulator) binds to PSCA which modifies the conformation of the allosteric regulator such that the inulin aptamer (e.g., the regulated aptamer) can bind to inulin.

### DETAILED DESCRIPTION OF THE INVENTION

Reference will now be made in detail to the presently preferred embodiments of the invention, which serve to explain the principles of the invention. It is to be understood that the application of the teachings of the present invention to a specific problem or environment will be within the capabilities of one having ordinary skill in the art in light of the teachings contained herein.

The present invention provides allosteric probes, which, in a preferred embodiment, include at least one allosteric regulator and at least one regulated aptamer. The allosteric regulator and the at least one regulated aptamer can each bind to a target.

The binding of the allosteric molecule to its target enhances the binding of the regulated aptamer to its target. The binding of the allosteric regulator to its target alters the conformation of the allosteric probe such that the binding of the at least one regulated aptamer to its target is permitted or enhanced resulting in a greater affinity between the regulated aptamer and its target than prior to the binding of the allosteric regulator to its target. The binding of the allosteric regulator to its target permits the allosteric probe to overcome the energy barriers to formation of an activated regulated aptamer.

In another embodiment, the binding of the allosteric regulator alters the conformation of the allosteric probe such that the binding of the at least one regulated aptamer to its target is inhibited or decreased. In this embodiment, the allosteric probe can, for example, shut off the activity of the regulated aptamer under certain conditions. For example, an allosteric probe designed to kill a tumor cell under a particular condition (e.g., the presence of a tumor marker) can be shut off in the presence of normal cell marker.

The allosteric probes of the invention have greatly increased specificity for their target because binding of the allosteric regulator to its target is preferably required to permit the regulated aptamer to bind to its target. Thus, non-specific interactions of the regulated aptamer and its target are greatly reduced. For example, nucleic acids and proteins non-specifically interact with a wide variety of molecules. While these non-specific interactions are generally of low affinity, they are generally much more abundant than specific interactions of a binding molecule and its target and thus can easily overwhelm a signal generated by a specific interaction (e.g., antibody/antigen interaction). For example, an antibody linked to an imaging or killing agent can bind non-specifically to many sites other than its intended target. In contrast, the preferred allosteric probes of the invention are highly sensitive, result in a higher signal to noise ratio, and are less susceptible to non-specific binding because the regulated aptamer preferably only binds to its target if the allosteric molecule binds to its target.

The term “binding,” as used herein, refers to the specific reaction of one or more molecules or compounds with respect to at least a second molecule or compound or group of molecules or compounds. The reaction may result in attachment (e.g., covalent bond) or association (e.g., ionic attraction) of the molecules with respect to each other. The term “specific reaction” is meant to indicate that the molecule or compound will react in a selective manner with its corresponding molecule or compound and not with a multitude of other molecules or compounds.

The term “enhanced binding” refers to an increased strength in the attraction or associations of one molecule to another molecule as measured, for example, by the dissociation constant, K_{d}, which is a measure of the relative concentrations of the components AB⇄A+B at equilibrium. A small K_{d} indicates stronger or more enhanced binding between two molecules (e.g., A and B). Preferably, the allosteric regulator is capable of enhancing the binding of the at least one regulated aptamer to its target only when the allosteric regulator is bound to its target.

The term “allosteric regulator” refers to a nucleic acid molecule (e.g., aptamer, and antisense molecule) with a function or activity that changes in response to the binding of an effector molecule (e.g., nucleic acid, protein, and/or chemical compound). For example, a nucleic acid molecule can be allosteric if it has at least two conformations: an active conformation, and an inactive conformation. Binding of the effector molecule to the allosteric molecule can shift the equilibrium between the inactive and active conformational states to favor the active conformation by making the active state more thermodynamically stable.

The terms “antagonist” or “inhibitor,” as used herein, refer to a molecule which, when bound to a target, blocks or modulates the biological or immunological activity of the target. Antagonists and inhibitors may include proteins, nucleic acids, carbohydrates, or any other molecules which bind to the target either covalently or non-covalently.

The term “antisense,” as used herein, refers to nucleotide sequences which are complementary to a specific DNA or RNA sequence. The term “antisense strand” means a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block further transcription or translation.

“Nucleic acid molecule,” as used herein, refers to an oligonucleotide, nucleotide, or polynucleotide, and fragments or portions thereof, and to DNA or RNA of genomic or synthetic origin which may be single- or double-stranded, and represent the sense or antisense strand.

As used herein, “regulated aptamer” refers to an aptamer whose activity is enhanced when an allosteric regulator is bound to the allosteric regulator's target. A regulated aptamer's activity can be enhanced, for example, by changing the conformation of the molecule resulting in an increased probability that the regulated aptamer will bind to its target.

In a preferred embodiment, the allosteric regulators of the present invention comprise any nucleic acid molecule or fragment capable of specifically binding to a target (e.g., nucleic acid, protein, antibody, carbohydrate, small molecule, and/or chemical compound). In one embodiment, the nucleic acid molecules are capable of folding into three-dimensional structures forming binding regions, pockets, and other secondary or tertiary surfaces on the molecule. Single-stranded nucleic acids can readily form three-dimensional structures. The binding sites of the molecules comprising the allosteric regulator can form highly specific complexes with particular target molecules. Examples of molecules that can be used in preferred embodiments of the invention include, but are not limited to, aptamers and antisense molecules.

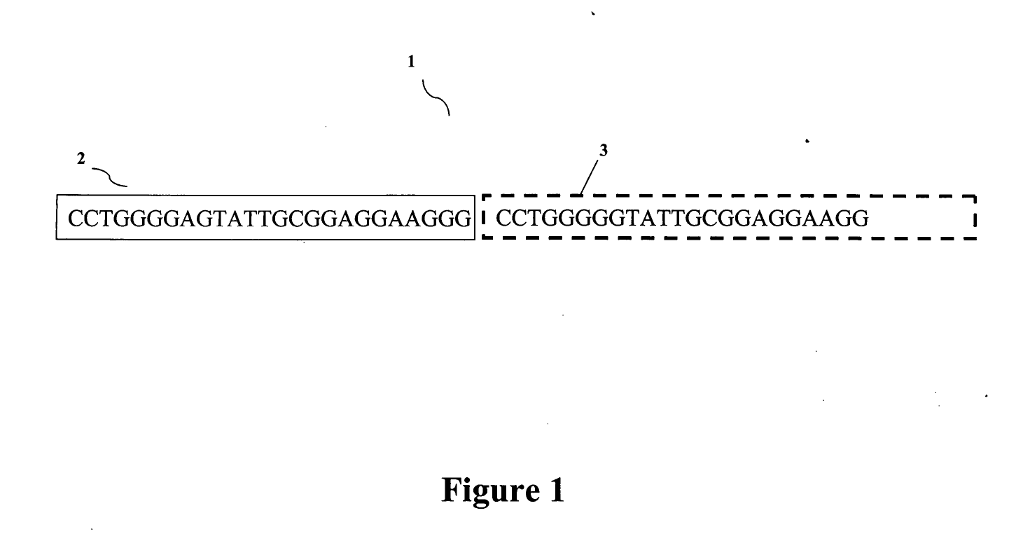

FIG. 1 illustrates an allosteric probe in accordance with a preferred embodiment of the invention. Allosteric probe 1 comprises allosteric regulator 2 and regulated aptamer 3. In a preferred embodiment, the components comprising allosteric probe 1 may be contiguous and cis-linked, as shown in FIG. 1. In this example, allosteric regulator 2 and regulated aptamer 3 are aptamers capable of binding to specific targets. Alternatively, the allosteric molecule and regulated aptamer can be oriented in other configurations (e.g., trans-configuration). In addition, the allosteric molecule and regulated aptamer can be non-contiguous (e.g., separated by a spacer molecule). In another embodiment, the allosteric probe may comprise a plurality of regulated aptamers each capable of binding to a specific target.

Alternatively, the allosteric probes comprise multiple molecules wherein, for example, the binding of one molecule to its target induces the binding of a second molecule to its target, which in turn induces the binding of a third molecule to its target in a “chain reaction” sequence. Each nucleic acid molecule in the exemplary allosteric probe can act as an allosteric molecule and regulate subsequent adjacent molecules. For example, the binding of an allosteric regulator to a first target regulates the ability of a regulated aptamer to bind its target. The binding of the regulated aptamer to its target can likewise regulate a further regulated aptamer to bind to its target.

In one embodiment of the invention, the allosteric molecule and/or the regulated aptamer is an aptamer capable of binding to a target molecule. Aptamers are nucleic acids that bind ligands with high specificity and high affinity and can be used, for example, to inhibit or interfere with the activity of nucleic acid or protein. Preferably, the affinity of an aptamer for its target is in the nanomolar range. Suitable aptamers can be selected using various techniques known in the art, such as “systematic evolution of ligands by exponential enrichment” or “SELEX.” Tuerk and Gold, Science 249, 505 (1990); Ellington and Szostak, Nature 346, 818 (1990), hereby incorporated by reference in their entirety. In one embodiment, the aptamer forms a stem-loop structure in which the bases in the loop form the binding region for interaction with a target.

Aptamers can be selected to bind to a variety of targets (e.g., proteins, nucleic acids, and compounds). For example, aptamers capable of binding proteins associated with a variety of diseases have been isolated. Sun, Curr Opin Mol Ther, 2(1), 100-105 (Feb. 2, 2000). These protein binding aptamers are used as antagonists to inhibit physiological functions associated with the protein target. Id. Aptamers capable of binding compounds such as theophylline, FMN, AMP, arginine, citrulline, tobramycin, and neomycin B have also been isolated and characterized. Hermann and Patel, Science, 287, 820-825 (2000). Aptamers can also be modified to increase their resistance to nucleases that may be encountered in cells or the bloodstream. Sun, Curr Opin Mol Ther, 2(1), 100-105 (Feb. 2, 2000). For example, modifying 2′ OH groups of the nucleotide bases of an aptamer to F or NH₂ increases the half-life of an aptamer in blood. Id. Alternatively, aptamers can be attached to higher weight delivery blood. Id. Alternatively, aptamers can be attached to higher weight delivery vehicles to increase their resistance to nucleases or antibodies. Aptamers can be delivered to cells by a variety of techniques including, but not limited to, expression vectors, liposomes, viral vectors, and cell fusion.

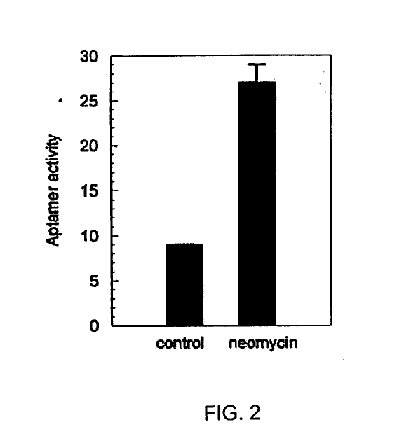

FIG. 2 shows the regulation of an exemplary allosteric probe comprising a neomycin aptamer (allosteric regulator) linked to an ATP aptamer (regulated aptamer). As shown in FIG. 2, the activity of the ATP aptamer, as measured by its ability to bind ATP, is enhanced approximately 3-fold in the presence of neomycin. Thus, the binding of the neomycin aptamer to neomycin regulates the binding of the ATP aptamer to ATP. This exemplary RNA allosteric probe has the following sequence: gcuuaauacgacucacuauaggccugggcgagaaguuuaggccuuggguugggaagaaacuguggcacuucggugccagcaaccc

Many aptamers have a stem-loop structure in which the bases in the loop and the stem are intimately involved in interaction with the ligand. Techniques for isolating single stranded DNA (ssDNA) provide a means of isolating ssDNA aptamers as well as RNA aptamers. DNA aptamers are capable of binding a wide variety of structures that include proteins and small molecules. Examples of ssDNA aptamers include aptamers that recognize thrombin, HIV reverse transcriptase, bile acids, cocaine, L-selectin, arginine, L-tyrosinamide and IgE. Kato, et al., Analyst 125:1371-3 (2000); Bock et al., Nature 355:564-6 (1992); Watson et al., Antisense Nucleic Acid Drug Dev 10:63-75 (2000); Wiegand et al., J Am Chem Soc 123:4928-31; Stojanovic et al., J Am Chem Soc 123:4928-31 (2001); Schneider et al., Biochemistry 34:9599-610 (1995); Robertson et al., Biochemistry 39:946-54 (2000); Vianini et al., Bioorg Med Chem 9:2543-8 (2001).

Several aptamers have been produced that recognize specific carbohydrate units. Wang, et al., Chem Biol. 2:281-90 (1995); Jiang, et al., Structure Fol Des 7:817-27 (1999). Some of the earliest selected aptamers were to the aminoglycosides, tobramycin and neomycin. Id.; Jiang, et al., Chem Biol 5:35-50 (1997). Electrostatic interactions with the amino groups on neomycin B and tobramycin contribute to the specific interaction of aptamer and aminoglycoside. Highly anionic aptamers can be useful for binding carbohydrates which consist of cationic sugars. In addition to ionic interactions, hydrogen bonding may be important in establishing aptamer-ligand interaction. For example, most of the contacts between neomycin B and its RNA aptamer, which binds through the major groove, are mediated by hydrogen bonding. Neutral and acidic sugars and other small molecules have also been successfully targeted for aptamer selection. The neutral glycolipid moenomycin has also been used for selecting a specific aptamer that binds to the moenomycin oligosaccharide with dissociation constants between 300 and 400 nM. Aptamers have also been selected for the Sialyl Lewis X blood group antigen, a structure that is also found in an H. Pylori LPS. Jeong et al., Biochem Biophys Res Commun 281:237-43 (2001).

Recognition that pathogenic bacteria are deadly, easily dispersed potential weapons for bioterrorism emphasizes the need for highly sensitive and effective diagnostic and therapeutic tools. Detection of pathogenic microbes in human tissue is especially difficult given the ability of pathogenic organisms to mimic normal cell surface markers making it difficult to distinguish the microbial lipopolysaccharide (“LPS”) in a background of human tissue. In addition, the facility with which bacteria mutate to produce a different strain with altered surface molecules, including altered LPS, requires the ability to rapidly design sensitive detection mechanisms adapted to the microorganism's defenses.

In a preferred embodiment of the invention, allosteric probes can detect the presence of pathogenic organisms by identifying the characteristic LPS on the surface of a suspected organism. For example, an allosteric probe can comprise an allosteric regulator which is capable of binding an LPS specific to a pathogenic organism (e.g., E. coli 157). The regulated aptamer of this exemplary allosteric probe can bind to a fluorescent marker target and catalyze a reaction which permits the aptamer to retain a signal. For example, a regulated aptamer target can be labeled with Tc^{99m} or F¹⁸ which is released after binding of the regulated aptamer to its labeled target. The location of the microorganism in vivo can be determined by autoradiography (e.g., for Tc^{99m} labeled target) or positron emission spectroscopy (e.g., for F¹⁸ labeled target). When the allosteric molecule binds to its LPS target, the regulated aptamer is activated, enhancing its ability to bind to its target and retain a detectable signal indicating the presence of the pathogenic bacteria.

Preferred allosteric probes of the invention can be used to diagnose diseases or conditions (e.g., cancer) by detecting the presence of a marker or markers. For example, an allosteric probe directed to HER2 can be used to specifically detect the presence of a breast tumor. An imaging agent can be used to provide detectable signals that can be detected by an imaging system (e.g., magnetic resonance imager) or other radiographic techniques.

The invention also provides therapeutic applications for allosteric probes. In a preferred embodiment, the allosteric molecule binds to a target cell (e.g., tumor cell, microorganism, or infected cell) and enhances the binding of the regulated aptamer to a drug (e.g., chemotherapeutic agent, prodrug, or antibiotic). In one embodiment, the allosteric molecule binds to a marker or other antigen at the surface of the target cell (e.g., receptor). The drug consequently is maintained and accumulates or is localized in proximity to the target cell. In another embodiment, the drugt can inactivate, damage, or kill the target cell.

In a preferred embodiment, the drug is inactive. After binding to the regulated aptamer, the prodrug selectively targets, is localized near, or is brought in proximity to the target cell. The target cell includes or secretes a marker (e.g., a target cell specific enzyme, a receptor, a carbohydrate moiety, or surface protein) which is capable of activating the prodrug to from a drug. The activated drug can carry out its intended activity on the target cell.

For example, allosteric probes of the invention can be used to treat prostate cancer by targeting cells expressing specific prostate cancer markers. Prostate cancer is the second leading cause of death by cancer of men in the United States, and it is the most frequently diagnosed form of cancer. In the United States in 2001, 198,100 men were diagnosed with prostate cancer and 31,500 died from the disease (American Cancer Society, Cancer Facts & Figures, 2001). Current therapies are clearly not adequate. Most current cancer therapies indiscriminately kill non-cancerous proliferating cells, including immune cells and hair follicle cells as well as cancer cells in what is termed the “bystander effect.” This heavy bystander cell loss is particularly serious because many of the affected cells are important for body defenses. Thus, accurate cell targeting is important for effective cancer therapy.

Allostery provided by the preferred allosteric probes of the invention reduces the concentration of the drug or prodrug near normal or bystander cells by reducing nonspecific interactions of the allosteric probe with other macromolecules that would lead to undesired high bystander cell killing. For example, an antibody tethered to a toxic agent can be used to deliver a toxic agent to a target cell. However, the antibody may have a high level of non-specific interactions with both target and normal cells resulting in an elevated bystander effect.

In contrast, the preferred allosteric probes of the invention must first bind the allosteric regulator to a target cell before the regulated aptamer can bind to its target. This preferred design permits highly specific and sensitive interactions with a minimum of background noise. The binding of the allosteric regulator to the target cell induces a conformational change which permits the regulated aptamer to bind to its target.

In a preferred embodiment, the target of the regulated aptamer is derivatized with a prodrug that is only activated by an enzyme secreted by a target cell or by the local pH or other special condition around or near the target cell membrane. Thus, when the allosteric regulator binds the target cell, the regulated aptamer binds the target molecule that is derivatized with a prodrug and brings the prodrug in the vicinity of the cell-produced modifier (e.g., cell surface antigen, locally secreted enzyme) which then, in turn, activates the prodrug. This exemplary design provides a significant improvement over the standard approach of tagging a monoclonal antibody with a toxic agent or a prodrug because the consequences of non-specific interactions (e.g., killing of bystander cells) are greatly reduced or eliminated.

A preferred target for the allosteric probes of the invention is the treatment of prostate cancer. Prostate stem cell antigen (PSCA) is a 123 amino acid, glycosylphosphoinositol-linked extracellular transmembrane protein member of the Ly6 protein family, and highly expressed on prostate cancers. Analysis of PSCA expression showed that it is associated with more than 91% of the 238 prostate cancers examined, including androgen-dependent and independent cancers. PSCA was also found in all 9 of the examined bone metastases. Consequently, PSCA has been identified as a target for cancer therapy and imaging and targeting antibodies are in development.

In one embodiment of the invention, an allosteric probe having an allosteric molecule capable of binding prostate-specific cancer antigen (PSCA) cis-linked to a regulated aptamer capable of binding inulin is provided. The allosteric probe binds inulin only in the presence of prostate-specific cancer antigen (PSCA). The inulin can be derivatized with a prodrug that can only be activated by prostate-specific antigen (PSA), a tumor specific protease that is produced at high levels by prostate cancer cells.

Cancer cells use proteases to cut a path for moving into and out of solid tissues. Therefore, unlike most normal tissues, metastatic tumors are surrounded by high concentrations of active proteases. Tumor-associated protease activity can be harnessed for killing tumor cells with a prodrug that is readily cleaved by the tumor protease. The prodrug itself is not toxic, but cleavage of the peptide bonds in the prodrug results in toxic products. Prostate specific antigen (PSA) is a secreted protease that is diagnostic for prostate cancer. PSA concentrations are high around metastasizing prostate cancers and the protease is active in fluids from these cancers. PSA has been targeted with peptide-based prodrugs linked with toxic agents, doxorubicin or thapsigargen. The prodrugs CPI-0004Na and L-377202 comprise peptides linked to doxorubicin.

In this embodiment, the allosteric molecule binds to PSCA on the surface of a prostate cancer cell and enhances the binding of the regulated aptamer to inulin derivatized with a prodrug. The prodrug is only activated in the presence of a protease such as PSA. If PSA is present, the prodrug is activated by cleavage of the peptide bonds with PSA. Thus, the prodrug is converted to an active drug which is located in close proximity to a prostate cancer cell (e.g., near the surface of the prostate cancer cell). The drug is highly unlikely to be activated in the proximity of normal cells which do not express PSCA or have high concentrations of proteases such as PSA. This exemplary allosteric probe is therefore highly specific for prostate cancer.

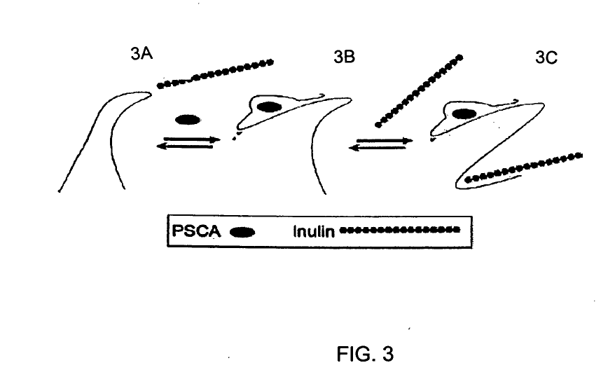

Referring to FIG. 3, an allosteric probe having a PSCA aptamer as an allosteric molecule and an inulin aptamer as a regulated aptamer is depicted. In FIG. 3, the PSCA aptamer binds to PSCA on the surface of a cell which induces a change in the conformation of the allosteric probe wherein the ability of the inulin aptamer to bind its target is enhanced (FIG. 3). FIG. 3 illustrates the binding of the inuling aptamer to inulin after the PSCA aptamer is bound to PSCA. Without being bound by theory, it is believed that if one aptamer (for PSCA) has a higher affinity for its target than the other (for inulin), then the presence of PSCA will drive the dynamic equilibrium of structural states toward a predominant state that includes the PSCA aptamer associated with PSCA as shown in FIG. 3. With the nucleic acid sequence that corresponds to the PSCA aptamer now removed from the dynamic possibilities for nucleic acid structures, there are significantly fewer possible structures for the remaining inulin aptamer sequence to adopt. As a result, the folded structure of the remaining nucleic acid is more likely to include the structure that binds inulin. The inulin aptamer structure can be further stabilized by the presence of inulin. Thus, the net result of the binding of PSCA is that the inulin aptamer can bind inulin.

Allosteric probes can be made to target a wide variety of therapeutic molecules including various cancer antigens (e.g., prostate-specific membrane antigen, PSCA, HER2, and epidermal growth factor receptor) and pathogenic microorganisms. For example, an allosteric probe for killing pathogenic microorganisms can be made by targeting LPS, an antibiotic (e.g., neomycin, tobramycin, penicillin, amoxicillin, and streptomycin) or other killing agent. In a preferred embodiment of the invention, an allosteric probe comprising an LPS aptamer or antibody is linked to an antibiotic aptamer or antibody (e.g., tobramycin aptamer). The binding of the LPS aptamer to its LPS target enhances the ability of the tobramycin aptamer to bind to tobramycin. Thus, tobramycin is localized in proximity to the surface of the pathogenic microorganism enhancing its killing effect.

It is to be understood that application of the present invention to a specific problem or environment will be within the capabilities of one having ordinary skill in the art in light of the teachings contained herein. Exemplary products of the present invention and processes for its use appear in the following Examples.

### EXAMPLE 1

### Identification and Isolation of LPS Aptamers

To prepare affinity columns, recombinant LPS (Sigma) is coupled to activated sepharose (Pharmacia) following standard procedures. The amount of each LPS coupled to the column is monitored by determining the difference between the LPS concentration in the starting buffer and in the buffer removed after coupling. Ninhydrin or rhodamine B is used to detect the LPS.

Examples of LPS structures that can be used as target molecules for selecting aptamer include 1) the 0:16 antigen of E. coli, and 2) the non-reducing terminal tetra or pentasaccharide region of the Haemophilus ducreyi lipooligosaccharide.

### Selection and Cloning of the LPS Aptamers (SELEX)

DNA aptamers will be selected by the SELEX procedure that was originally described for selecting RNA aptamers but that has since been modified for selecting DNA aptamers. A mixture of randomly varied single-stranded DNAs is passed multiple times through an LPS-sepharose column with intervening amplifications steps that also introduce new variations into the sequence. To make aptamers that recognize specific LPS O-specific oligosaccharides, a pool of single stranded DNA containing about 10¹⁴ random sequences is used. The structure of each of DNA molecule includes a 40 nucleotide core of random DNA sequence surrounded by terminal invariable sequences for PCR amplification. With each successive cycle, the selection condition is made incrementally more rigorous by decreasing the salt concentration in the loading buffer and increasing the temperature of the buffer.

Procedures for making ssDNA aptamers have been used to isolate ssDNA aptamers. Several procedures can be used to prepare the ssDNA including asymmetric PCR or PCR amplification using a 5′ or 3′primer with a ribose at the 3′ end or using a 5′ biotinylated primer. ssDNA can be isolated by electrophoresis or by streptavidin affinity capture, depending on the preparation.

The O-specific polysaccharide portion of LPS is used to elute the LPS-specific ssDNAs from the affinity column. In this way, KDO-specific and lipid A specific aptamers are not eluted. To increase the probability of obtaining aptamers of high affinity, elutions are preceded by incubating a closed column for 30 minutes with the ssDNA mixture followed by extensive washing and elution of the bound aptamers.

After the first few rounds of selection, the KDO-lipid A portion of the LPS is preincubated with the aptamer pool before it is passed through the column. The aptamers that are bound to the LPS in the presence of competing KDO-lipid A are depleted in the aptamer pool. In these rounds of selection O-specific polysaccharides are eluted in hot (95° C.) buffer. This procedure allows the retention of very high affinity aptamers in the selected pool. For the H. ducreyi O-specific oligosaccharide, the ssDNA pool is preincubated with human blood group antigen, lacto-N-neotetraose after about 3 and 6 rounds of selection to remove aptamers that recognize this oligosaccharide.

After each round of selection, the pool size of the DNA in the mixture is monitored by determining the percent of total loaded ssDNA that is adsorbed and eluted from the affinity column. The selection process is stopped when there is no increase in the fractional capture of the loaded ssDNA (measured using radiolabeled DNA), typically after 6-12 cycles.

The ssDNA candidates are cloned and sequenced to identify consensus sequences for aptamer structure. Consensus sequence(s) will be synthesized chemically and tested for the ability to bind to LPS-sepharose and for the ability to be eluted by LPS, the O-specific oligosaccharide, the KDO-lipid A moiety, and the Ra antigen. Similar tests can be done by affinity chromatography to determine the ability to bind LPS and the ability of the various LPS fragments to inhibit binding. Aptamers that specifically bind to LPS and to the target O-specific polysaccharide and not to the KDO and core polysaccharide, lipid A, or alternate LPS O-specific polysaccharide are identified for further studies.

### Characterization of the Selected Aptamers for the LPS

The affinities and specificities of the LPS aptamers will be determined for their O-specific oligosaccharide targets. For this determination we use ³²P-labeled aptamers, biotinylated LPS and streptavidin coated magnetic beads. The streptavidin-coated beads can used to capture and thus measure the amount of LPS-bound aptamer under each equilibrium condition. In preliminary studies, conditions are sought to maximize interaction of the aptamer to the LPS by varying the concentration of salt, protons (pH) and divalent cations. ATP-specific DNA aptamer that should have no specific affinity for LPS can be used as negative controls. Conditions are optimized for binding of the LPS aptamer and against nonspecific binding of the ATP aptamer to the LPS. To achieve high concentrations of monomers, LPS can be solubilized in 0.2% deoxycholate. The time of incubation with LPS can be varied in these preliminary studies to determine the time required to reach equilibrium. To measure aptamer affinity for various LPS molecules and constituents, the radiolabeled aptamer can be first incubated at 4° C. for the determined time to equilibrium with biotinylated LPS and various concentrations of individual competing O-specific polysaccharides, KDO-lipid A, and other polysaccharides. The amount of aptamer that remains bound to the LPS can be determined after its removal on the streptavidin beads and several washes under binding conditions (the number and volume of washes will also be optimized).

The amount of bound aptamer can be measured and, in experiments in which the concentration of aptamer is varied with a constant concentration of LPS, the number of aptamer molecules bound can be measured using a Scatchard analysis. This analysis can be used to determine the average maximum number of binding sites for the aptamer per LPS molecule. With this knowledge and the specific activity of the ³²P-labeled aptamer, the Ki for each competitor can be determined. From these results, the affinity of each aptamer for the LPS against which it was targeted can be determined. The specificity of the aptamer for its target LPS and for the O-specific oligosaccharide that makes up the target can also be determined.

If the selected aptamers are of low affinity for their LPS targets. the selection procedure will be initiated with a new pool of random DNA sequences. In addition, a “doping” procedure can be used to select an aptamer with desirable characteristics. “Doping” is directed to chemically synthesizing a library of aptamers where a predetermined percentage each nucleotide base is replaced with the three nucleotide bases not normally present in that position. For example, an A at position one would be replaced with T, C, and G in a predetermined percentage of aptamer sequences. Doping introduces variability into a library of aptamer sequences providing a pool from which a desired aptamer can be selected.

### Development of an Allosteric LPS Aptamer

An allosteric probe can be developed having an LPS aptamer as an allosteric regulator of a regulated aptamer used to mark the presence of the targeted LPS. An allosteric probe is formed from a high affinity LPS for the allosteric regulator and a lower affinity aptamer for the regulated aptamer. An ATP aptamer can be used as the lower affinity, regulated aptamer. The affinities of the two aptamers are preferably at least a log unit apart. Low affinity aptamers can have, for example, a dissociation constant of more than 100 micromolar.

The allosteric probe can be further enhanced by selection after doping in a procedure that will involve two affinity columns in a series. The allosteric probes are passed through the first affinity column that contains the regulated aptamer ligand so as to remove those sequences that already bind to this ligand in the absence of the allosteric regulator ligand. The allosteric probes that successfully pass through this affinity column are then incubated with the allosteric regulator ligand and are passed through a second affinity column of the regulated aptamer ligand. This time, regulated by the allosteric regulator, the allosteric probes with the highest activity will be bound to the column.

From these studies, aptamer pairs included together in a allosteric probe behave as an allosteric pair with one aptamer regulating the activity of the other. The allosteric probe can be used to detect LPS in a homogeneous assay format.

### EXAMPLE 2

### RNA Systematic Evolution of Ligands by Exponential Enrichment (Selex) for Isolating RNA Aptamers

The RNA SELEX procedure identifies desired RNA aptamers from a pool of randomly varied single-stranded DNA molecules which have invariant ends containing an annealing site for a Klenow primer, an annealing site for T7 RNA polymerase promoter, and polymerase chain reaction (PCR) primer sites.

The Klenow primer provides the priming site for the Klenow fragment of DNA polymerase I, which converts the single-stranded pool into a random pool of double-stranded DNA. The random pool of double-stranded DNA molecules serve as templates for the T7 RNA polymerase to generate a random pool of RNA molecules which can be used for selection of RNA aptamers using. The random RNA pool can be reacted a desired target (e.g., LPS, tumor cell market) in a binding reaction using 10¹⁵ RNA molecules and 10¹⁴ target molecules for a 10:1 molar ratio. Unbound RNA molecules can be removed by partitioning, usually on nitrocellulose filters.

The selected RNA can be subjected to a further round of selection according to the following steps. First, reverse transcriptase can be applied to the selected RNA to produce a complementary DNA (cDNA). Next, Taq DNA polymerase is used to amplify the cDNA in a PCR reaction and produce a double-stranded DNA. The double-stranded DNA can serve as a template for T7 RNA polymerase, which generates the amplified pool of selected RNA, to be used in the second round of selection. This cycle is repeated until a single RNA aptamer is produced or isolated.

### EXAMPLE 3

### PSCA Allosteric Clamp

An exemplary allosteric CLAMP comprises two cis-linked aptamers in which the binding of a target to one aptamer regulates the activity of the other (FIG. 3). The exemplary allosteric CLAMP: 1) targets a cell surface protein (PSCA) that is more highly expressed on tumor cells than on normal cells; 2) uses an aptamer with high affinity and specificity for PSCA; 3) uses a peptido prodrug that is selectively cleaved by a tumor-derived protease; and 4) links the prodrug to the second target of a bifunctional aptamer. Thus, the allosteric CLAMP's molecular configuration brings together two approaches to targeting cancer and will allow a synergy between PSA and PSCA in which the PSCA-CLAMP will concentrate the prodrug around the tumor cells for PSA to degrade the drug. The resulting synergy requires lower doses of prodrug for killing tumors and bystander cell death will be less than with the prodrug alone.

The peptido-prodrug is linked to inulin, a polysaccharide made up of repeating fructan units (2,1 fructofuranan) which is used currently in medical applications for evaluating kidney function. The inulin preparations used for intravenous injections into humans are about 30 sugar units in length. These chains are flexible and can be produced as shorter or longer units. Inulin can be derivatized with peptides and proteins. A single inulin molecule can carry at least one peptido prodrug per sugar. The approximate 300D length of the 30-unit inulin molecule will stretch a considerable distance from the cell surface where it is linked to PSCA via the CLAMP. This configuration will provide a 300D thick space around the cancer cell where the prodrug is concentrated for PSA cleavage. The inulin binding activity of the aptamer is chosen such that the CLAMP has a high on and off rate to increase the exchange rate of inulin and inulin-prodrug complex in the vicinity of the cell. This will allow the rapid exchange of cleaved target molecule and new uncleaved target molecule with attached prodrug to be exposed to PSA over the entire treatment period.

In one example, the allosteric CLAMP is generated using a mixture of about 10¹⁴ DNA sequences that contain a defined 5′ sequence for PCR amplification, a stretch of 40 randomized bases, and the PSCA aptamer sequence at the 3′ end. RNAs transcribed from these sequences are selected for their ability to bind to inulin in the presence but not in the absence of PSCA. The resulting RNA sequences (allosteric CLAMPs) are further characterized for their ability to bind inulin in response to PSCA and for their ability to selectively target cells that express high PSCA.

To prepare affinity columns, recombinant PSCA is coupled to activated sepharose (Pharmacia) following standard procedures. Inulin affinity columns are prepared from agarose adipic acid hydrazide (Pharmacia). The amount of inulin linked to the column can be determined by measuring the difference between the initial amount inulin and coupled inulin. PSCA coding sequence (amino acids 16-123) can be subcloned into the pGEX plasmid for expression in the Origami B(DE3)pLys S strain of E. coli (Novagen) increasing the likelihood of proper folding of the extracellular eukaryotic proteins due to the presence of an oxidizing intracellular environment. An oxidizing intracellular environment encourages folding of highly disulfide-linked extracellular proteins.

The PSCA protein can be subcloned with an amino terminal histidine tag separated from the mature protein by an enterokinase cleavage site and a carboxy terminal GST tag separated from the mature protein by a thrombin cleavage site. These two tags can be used to purify the protein by metal chelate chromatography and GST affinity chromatography. The tags can be removed by treatment with the appropriate proteases before using the protein to select for aptamers.

Alternatively, if PSCA is not well expressed in E. coli or if the resulting protein is insoluble, the protein can be expressed in mammalian cells with a secretory signal to ensure extracellular delivery. Previously, PSCA has been successfully expressed in mammalian cells. We have constructed a mammalian expression cassette and used it successfully for the production of two secreted proteins by human kidney 293 cells. The cassette (containing a CMV promoter, an actin promoter, and an SV40 polyadenylation signal) produces a protein with an alkaline phosphatase signal sequence followed by a polyhistidine tag and an enterokinase site resulting in cleavage between the poly-his tag and the first amino acid of the native protein. The construct can be used to stably transfect 293 cells and isolate high expressing clones. The protein can be expressed from the conditioned medium of these cells using nickel chelate and glutathione columns for purification.

Inulin can be functionalized with peptides as previously described. Fluorescein isothiocyanate can be used to label the peptides to produce a fluorescent-labeled inulinpeptide target. The human cancer cell line RT112 expresses very low levels of PSCA mRNA and is stimulated by phorbol esters to produce very high levels of PSCA. These cells can be used for selection of the aptamer against PSCA on the cell surface. The level of PSCA protein expression in the presence and absence of phorbol ester stimulation can be confirmed by Western blotting using two goat anti-human PSCA antibodies, which have been demonstrated to be effective in Western blotting (sc-14201, sc-14202; Santa Cruz Biotechnology).

Allosteric CLAMPs can be produced by selecting for RNAs that bind inulin in response to PSCA. To make CLAMPs that recognize inulin only in the presence of PSCA, a pool of single stranded DNA containing about 10 random sequences can be used. The selection procedure involves the repeated selection of the desired property from a large pool of nucleic acid sequences. The starting ssDNA sequence can be constructed as follows: (a) from 5′-3′ using a defined sequence for the 5′ primer; (b) followed by 40 random bases; and (c) followed by a 20 base sequence for PCR amplification. For PCR amplification, the 5′ primer can also include a T7 promoter. The sequence of the 5′ primer can be adjusted to be compatible in PCR with the 3′ primer. The initial buffer conditions to be used for the selection include Hanks balanced salts solution without glucose (HBSS; 0.137 M NaCl, 5.4 mM KCl, 0.22 mM Na₂HPO₄, 0.44 mM KH₂PO₄, 0.4 mM MgSO₄, 1.26 mM CaC₂, 0.49 mM MgC₂, 4.16 mM NaHCO₃). The initial buffer conditions were chosen as a baseline to approximate the salt concentration and pH in the tissues in which PSCA is found on the cell surface. We can also determine that, under the chosen buffer conditions, PSCA does not bind to the inulin affinity column. If binding is observed, the buffer conditions can be varied to minimize this binding. Transcription using T7 polymerase can be done with 2′fluoro-pyrimidine nucleotide triphosphate substrates (Epicentre). These modified pyrimidines were used in selecting the PSMA aptamer and have been shown to result in highly stable RNA molecules that can be used in vivo. RNA is one preferred nucleic acid for making aptamers because the additional hydroxyl group at the 2′ position provides more options for specificity and affinity of target binding. RNA aptamers that bind to inulin are preferably selected in the presence, but not in the absence, of PSCA.

Aptamers used in the CLAMPs can be selected using the following steps: 1) selection of RNAs that do not bind to the inulin affinity column in the absence of PSCA; 2) selection of RNAs resulting from step 1, on a second inulin affinity column of RNAs that binds inulin in the presence of PSCA; and 3) elution by inulin from the second column of those aptamers that bind inulin. After each round, the pool size of the DNA in the mixture can be monitored by determining the percent of total loaded RNA that is adsorbed and eluted from the affinity column. Selection can be stopped (e.g., after about 6-12 cycles) when there is no increase in the fractional capture of the loaded RNA as measured by real-time PCR of the reverse transcribed cDNA.

The aptamer can also be selected by its ability to bind to PSCA on the cell surface and not to bind other cell surface proteins by performing a negative selection against RT112 cells (i.e., cells that express low or no PSCA) preincubated with anti-PSCA antibody to occlude any remaining PSCA antigen on the cell surface. Positive selection can then be applied to identify aptamers that bind phorbol ester stimulated RT112 cells, which express high levels of PSCA. The selection processes against PSCA on the cell surface can be done after every three rounds of selection through the affinity column. Prior to incubation with the nucleic acids, the cells to be used for selection of aptamers can be washed and incubated at 4° C. for 30 minutes, and incubated for 15 min at 37° C. with RNA in order to remove extracellular nucleases and prevent the cells from releasing additional nucleases. Additional selection steps can be applied using cells that have not been first incubated at 4° C. in order to remove, from the selected population, those aptamers that bind PSCA. conformers created by a transient shift to 4° C.

The selected aptamer RNAs for PSCA-regulated inulin binding can be cloned and sequenced to identify consensus sequences for aptamer structure. Serially truncated versions of the resulting CLAMPs can be obtained by varying the PCR primers for amplification. The resulting RNA sequence(s) can be synthesized and tested for the ability to bind inulin under the control of PSCA. The CLAMPs will first be tested for their abilities to bind to an inulin-agarose affinity column in the presence and the absence of PSCA and for their abilities to be eluted from the column by inulin. Similar tests will be done by electrophoretic mobility shift assay (EMSA), with fluorescent peptido-inulin, to determine the ability of the clamp to bind inulin in the presence and absence of PSCA. From these studies, we expect to have identified an allosteric CLAMP that binds PSCA and inulin such that PSCA is an allosteric regulator of inulin binding.

The resulting allosteric CLAMPs can be tested at the cell surface of RT112 cells that have and have not been activated by phorbol esters in order to analyze the specificity of the interaction between the allosteric CLAMP and the cells. The CLAMP can be labeled with rhodamine (Molecular Probes) to visualize binding of the CLAMP to cell surfaces. The amount of fluorescence per cell can be determined quantitatively by image analysis (ISU Image Analysis Facility) to determine the amount of rhodamine-labeled allosteric CLAMP associated with at least one hundred randomly selected cells. Fluorescein-labeled inulin can also be included in the medium with the CLAMP to determine the amount of inulin associated with cells that express PSCA compared with those that do not express PSCA. Control RNA with a scrambled sequence can be used to determine the nonspecific background binding ability of the CLAMP to the cells.

For each of the experimental conditions the average fluorescence (rhodamine or fluorescein) over each cell will be determined; where FH and Fc are the average fluorescent emissions/cell (F) for phorbol-ester stimulated RT112 cells (H) and control, untreated RT112 cells (c) respectively. The specificity of labeling can be obtained from the calculation: (F_{H}−F_{C})/F_{C}. Control studies can be included in each experiment to establish the level of expression of PSCA protein in each cell culture by quantitative Western blot. A standard curve can be established by varying the amount of recombinant PSCA protein loaded on the gel.

The above description is only illustrative of preferred embodiments which achieve the objects, features, and advantages of the present invention, and it is not intended that the present invention be limited thereto. Any modifications of the present invention which come within the spirit and scope of the following claims is considered part of the present invention.

1. A probe for binding a plurality of targets comprising an allosteric regulator linked to at least one regulated aptamer wherein binding the allosteric regulator with a first target enhances the binding of the at least one regulated aptamer to at least one second target.

2. A probe for binding a plurality of targets comprising an allosteric regulator linked to at least one regulated aptamer wherein binding the allosteric regulator with a first target inhibits the binding of the at least one regulated aptamer to at least one second target.

3. The probe of claim 1, wherein the allosteric regulator and the at least one regulated aptamer are linked in a cis configuration.

4. The probe of claim 1, wherein the binding of the allosteric regulator for the first target has a first affinity and the binding of the at least one regulated aptamer for the at least one second target has a second affinity, and the first affinity is greater than the second affinity.

5. The probe of claim 1, wherein the allosteric regulator comprises a tobramycin aptamer and the at least one regulated aptamer comprises an ATP aptamer.

6. The probe of claim 1, wherein the allosteric regulator is a nucleic acid molecule.

7. The probe of claim 6, wherein the nucleic acid molecule is selected from the group consisting of an antisense molecule and an aptamer.

8. The probe of claim 7, wherein the aptamer is capable of binding targets selected from the group consisting of bitheophylline, FMN, AMP, arginine, citrulline, tobramycin, ATP, and neomycin B.

9. The probe of claim 1, wherein the first target is selected from the group consisting of prostate specific antigen, prodrug, a tumor cell, receptor, carbohydrate, lipopolysaccharide, and oligosaccharide.

10. The probe of claim 1, wherein the at least one second target is selected from group consisting of prostate specific antigen, a drug, a prodrug, a tumor cell, receptor, carbohydrate, lipopolysaccharide, and oligosaccharide.

11. The probe of claim 9, wherein the prodrug is selected from the group consisting of CPI-0004Na and L-377202.

12. The probe of claim 9, wherein the prodrug is a peptide-based prodrug.

13. The probe of claim 10, where the prodrug is selected from the group consisting of CPI-0004Na and L-377202.

14. The probe of claim 10, where the prodrug is a peptide-based prodrug.

15. The probe of claim 10, where the oligosaccharide is an O-specific oligosaccharide.

16. The probe of claim 15, wherein the O-specific oligosaccharide is selected from the group consisting of the O58 and O124 polysaccharides of E. coli.

17. The probe of claim 1, wherein the at least a second target is an imaging agent selected from the group consisting of a fluorescent molecule and a radioactive molecule.

18. The probe of claim 1, wherein the at least one second target is a toxic agent.

19. A method of detecting a plurality of targets comprising, providing an allosteric probe containing an allosteric regulator linked to at least one regulated aptamer, contacting the probe with a first target, wherein the binding of the allosteric regulator to the first target enhances the binding of the regulated aptamer to at least the second target and produces a detectable signal.

20. The method of claim 19, wherein the at least one second target comprises an imaging agent selected from a group consisting of fluorescent molecule and a radioactive molecule.

21. The method of claim 20, wherein the detectable signal is generated by retention of the fluorescent molecule.

22. The method of claim 20, wherein the fluorescent molecule is selected from the group consisting of Tc^{99m} and F¹⁸.

23. The method of claim 22, wherein the first target is a microorganism.

24. The method of claim 23, wherein the location of the microorganism is determined by measuring the location of the detectable signal.

25. The method of claim 24, wherein the location of the detectable signal is measured by autoradiography or positron emission spectroscopy.

26. The method of claim 19, wherein the detectable signal indicates the presence of prostate stem cell antigen.

27. The method of claim 26, wherein the detectable signal indicates the presence of prostate specific antigen.

28. The method of claim 19, wherein the detectable signal indicates the presence of lipopolysaccharide.

29. The method of claim 19, wherein the allosteric regulator and the at least one regulated aptamer are linked in a cis configuration.

30. The method of claim 19, wherein the affinity of the allosteric regulator for the first target is higher than the affinity of the regulated aptamer for the second target.

31. The method of claim 19, wherein the allosteric regulator comprises a nucleic acid molecule.

32. The method of claim 19, wherein the first target is selected from group consisting of prostate specific antigen, a drug, a prodrug, a tumor cell, a microorganism, a receptor, carbohydrate, lipopolysaccharide, and oligosaccharide.

33. The method of claim 19, wherein the at least a second target is selected from group consisting of prostate specific antigen, a drug, a prodrug, a tumor cell, a microorganism, a receptor, carbohydrate, lipopolysaccharide, and oligosaccharide.

34. The method of claim 32, wherein the prodrug is selected from the group consisting of CPI-0004Na and L-377202.

35. The method of claim 32, wherein the prodrug is a peptide-based prodrug.

36. The method of claim 32, wherein the oligosaccharide is an O-specific oligosaccharide.

37. The method of claim 33, wherein the prodrug is selected from the group consisting of CPI-0004Na and L-377202.

38. The method of claim 33, wherein the prodrug is a peptide-based prodrug.

39. The method of claim 33, where the oligosaccharide is an O-specific oligosaccharide.

40. The method of claim 19, wherein the at least one second target is an imaging agent selected from the group consisting of a fluorescent molecule and a radioactive molecule.

41. The method of claim 19, wherein the at least one second target is a toxic agent.

42. A method of selectively targeting a drug or prodrug to a target cell, comprising providing an allosteric probe containing an allosteric regulator and a regulated aptamer, contacting the probe with the target cell, wherein the contacting of the allosteric regulator with the target cell enhances the binding of the regulated aptamer to a toxic agent to selectively target the drug or prodrug to the target cell.

43. The method of claim 42, where the drug or prodrug inactivates the target cell.

44. The method of claim 42, wherein the drug or prodrug is selected from the group consisting of a chemotherapeutic agent, an antibody, and an antisense molecule.

45. The method of claim 42, wherein the target cell is a tumor cell.

46. The method of claim 42, wherein the allosteric regulator and the at least one regulated aptamer are linked in a cis configuration.

47. The method of claim 42, wherein the binding of the allosteric regulator for the target cell has a first affinity and the binding of the regulated aptamer for the second target has a second affinity, and the first affinity is greater than the second affinity.

48. The method of claim 42, wherein the allosteric regulator comprises a nucleic acid molecule.

49. The method of claim 48, wherein the nucleic acid molecule is selected from the group consisting of an antisense molecule and an aptamer.

50. A method of selectively targeting a tumor inhibiting drug to a prostate stem cell antigen expressing cell, comprising, contacting the prostate stem cell antigen expressing cell with an allosteric probe comprising an allosteric regulator capable of binding to the prostate stem cell antigen expressing cell and a regulated aptamer capable of binding to a prodrug which is activated to a drug in the presence of prostate specific antigen, wherein the contacting of the allosteric regulator with the prostate stem cell antigen expressing cell enhances the binding of the regulated aptamer to prodrug, causes the activation of the prodrug to the drug in the presence of prostate specific antigen, and selectively targets the drug to the prostate stem cell antigen expressing cell.

51. The method of claim 50, wherein the prodrug inactivates the prostate stem cell antigen expressing cell.

52. The method of claim 50, wherein the prodrug is selected from the group consisting of CPI-0004Na and L-377202.

53. A method of selectively targeting an antibiotic to a microorganism susceptible to the antibiotic comprising contacting the microorganism with an allosteric probe comprising an allosteric regulator capable of binding to the microorganism and a regulated aptamer capable of binding to the antibiotic, wherein the contacting of the allosteric regulator with the microorganism enhances the binding of the regulated aptamer to the antibiotic and selectively targets the antibiotic to the microorganism.

54. The method of claim 53, wherein the antibiotic inactivates the microorganism.

55. The method of claim 53, wherein the antibiotic is selected from the group consisting of neomycin, tobramycin, penicillin, amoxicillin, and streptomycin.
